(19) United States
(12) Patent Application Publication
(10) Pub. No.: US 2016/0089040 A1
(43) Pub. Date: Mar. 31, 2016

(54) BIOLOGICAL INFORMATION DETECTING DEVICE

(71) Applicant: Seiko Epson Corporation, Tokyo (JP)

(72) Inventors: Atsushi Narusawa, Chino-shi (JP); Shinichiro Watanabe, Koto-ku (JP)

(21) Appl. No.: 14/860,753

(22) Filed: Sep. 22, 2015

(30) Foreign Application Priority Data
Sep. 29, 2014 (JP) 2014-198240
Sep. 29, 2014 (JP) 2014-198241

(51) Int. Cl. A61B 5/024; A61B 5/0205; A61B 5/11; A61B 5/1455

(52) U.S. Cl. CPC A61B 5/02405; A61B 5/14551; A61B 5/0205; A61B 5/1117

(57) ABSTRACT

A biological information detecting device includes: a pulse wave information measuring unit that measures pulse wave information of a subject; an event determination unit that determines whether or not a syncope event including a sign of syncope and development of syncope has occurred in the subject based on the measured pulse wave information; and a control unit that, when it is determined that the syncope event has occurred, performs at least one of storage processing of the pulse wave information at the time of the occurrence of the syncope event and the syncope event notification processing.

### CROSS-REFERENCE

This application incorporates the contents of Japanese Patent Application No. 2014-198240, filed on Sep. 29, 2014 and Japanese Patent Application No. 2014-198241, filed on Sep. 29, 2014 by reference.

### BACKGROUND

1. Technical Field

The present invention relates to a biological information detecting device or the like.

2. Related Art

According to the diagnosis and treatment guidelines (2012 revised version) of syncope, in a survey of 3,877 syncope patients in the United States, it has been found that 381 patients (9.8%) had a syncopal attack while driving. Syncope during the driving is very likely to immediately lead to an accident. Therefore, syncope during the driving is dangerous. According to the guidelines described above, it has been found that patients who had a syncopal attack while driving are relatively young men and many of them are patients with a history of cardiovascular disease, compared with patients who have not experienced a syncopal attack while driving even if there is a history of syncope. Therefore, it is important to monitor the development of syncope and rightly diagnose the cause of syncope early. As an invention regarding the detection of the cause of syncope, there is a known technique disclosed in JP-T-2011-505891. JP-T-2011-505891 proposes a method of monitoring the state of the subject at the time of syncope by using a patched type ECG (electrocardiogram), which can be easily mounted on the subject, and a pulse wave.

As the cause of syncope, there are orthostatic, reflective (neurogenic), and cardiogenic. Among these, in the case of cardiogenic syncope, if there is a suspicion of arrhythmia, an implantable electrocardiograph is used. In this case, however, it is necessary to implant the electrocardiograph under the skin. Accordingly, since the burden on the patient is large, many patients refuse the implantation of the electrocardiograph even if a doctor recommends the implantation of the electrocardiograph.

For this reason, the method disclosed in JP-T-2011-505891 has been proposed.

### SUMMARY

An aspect of the invention relates to a biological information detecting device including a pulse wave information measuring unit that measures pulse wave information of a subject; an event determination unit that determines whether or not a syncope event including a sign of syncope and development of syncope has occurred in the subject based on the measured pulse wave information; and a control unit that performs at least one of storage processing of the pulse wave information at the time of occurrence of the syncope event and notification processing of the syncope event when it is determined that the syncope event has occurred.

Another aspect of the invention relates to a biological information detecting device including a pulse wave information measuring unit that measures pulse wave information of a subject; and a control unit that calculates syncope index information, which changes according to a situation of a sign of syncope or development of syncope in the subject, based on the measured pulse wave information, in which the control unit calculates the syncope index information based on pulse wave amplitude information of a plurality of pulsations within a given period of time.

### BRIEF DESCRIPTION OF THE DRAWINGS

The invention will be described with reference to the accompanying drawings, wherein like numbers reference like elements.

FIG. 1 is an example of the system configuration of the present embodiment.

FIGS. 2A to 2D are explanatory diagrams of a pulse waveform when there is a sign of syncope.

FIGS. 3A to 3E are explanatory diagrams of syncope index information.

FIG. 4 is an explanatory view of a pulse coefficient.

FIG. 5 is a flowchart illustrating the flow of the process of the present embodiment.

FIG. 6 is a flowchart illustrating the flow of syncope event detection processing.

FIG. 7 is a flowchart illustrating the flow of notification processing.

FIG. 8 is a flowchart illustrating the flow of initial setting.

FIG. 9 is another flowchart illustrating the flow of the syncope event detection processing.

FIG. 10 is a flowchart illustrating the flow of processing for comparison with a first event pulse.

FIG. 11 is another flowchart illustrating the flow of the notification processing.

FIG. 12 is a flowchart illustrating the flow of syncope index information specification processing.

FIGS. 13A and 13B are external views of a biological information detecting device of the present embodiment.

FIG. 14 is an external view of the biological information detecting device of the present embodiment.

FIG. 15 is a diagram for explaining the mounting of a biological information detecting device and communication with an information processing apparatus.

FIGS. 16A and 16B are explanatory diagrams of a display image.

### DESCRIPTION OF EXEMPLARY EMBODIMENTS

In the method disclosed in JP-T-2011-505891, however, it is necessary to attach an electrode to the chest for the patch type ECG similar to a normal ECG. Since the skin may be irritated if the patch type ECG is attached for a long time, the method disclosed in JP-T-2011-505891 has not been suitable for long-time measurement.

According to some aspects of the invention, a biological information detecting device and the like capable of detecting a sign of syncope or development of syncope while suppressing the physical burden on a subject can be provided.

Also, according to some aspects of the invention, a biological information detecting device and the like capable of outputting to the viewer that there has been the sign of syncope or syncope has been developed as more understandable information can be provided.

In the present embodiment, pulse wave information is measured, and it is determined whether or not a syncope event including the sign of syncope and the development of syncope has occurred in the subject based on the measured pulse wave information. In addition, when it is determined that a syncope event has occurred, at least one of the storage processing of the pulse wave information at the time of the occurrence of the syncope event and the syncope event notification processing is performed.

Therefore, it is possible to detect the sign of syncope or the development of syncope while suppressing the physical burden on the subject.

In the present embodiment, when it is determined that the subject is in a bradycardia state based on the pulse wave information, an event determination unit may determine that the syncope event has occurred in the subject.

Therefore, it is possible to store the pulse wave information when the subject is in a bradycardia state or to send notification indicating that the subject is in a bradycardia state, for example.

In the present embodiment, when a pulse rate specified based on the pulse wave information is less than a first threshold value, the event determination unit may determine that the subject is in a bradycardia state.

Therefore, by adjusting the first threshold value, it is possible to adjust the state of the subject determined to be in a bradycardia state.

In the present embodiment, when it is determined that the subject is in a tachycardia state based on the pulse wave information, the event determination unit may determine that the syncope event has occurred in the subject.

Therefore, it is possible to store the pulse wave information when the subject is in a tachycardia state or to send notification indicating that the subject is in a tachycardia state, for example.

In the present embodiment, when a pulse rate specified based on the pulse wave information is equal to or greater than a second threshold value and a pulse wave amplitude at a first timing specified based on the pulse wave information is greater than a pulse wave amplitude at a second timing after the first timing, the event determination unit may determine that the subject is in the tachycardia state.

Therefore, by adjusting the second threshold value, it is possible to adjust the state of the subject determined to be in a tachycardia state.

In the present embodiment, when it is determined that the subject is in a pulse missing state based on the pulse wave information, the event determination unit may determine that the syncope event has occurred in the subject.

Therefore, it is possible to store the pulse wave information when the subject is in a pulse missing state or to send notification indicating that the subject is in a pulse missing state, for example.

In the present embodiment, when a pulse rate specified based on the pulse wave information is equal to or greater than a third threshold value and less than a fourth threshold value and no pulse is detected over a given period of time in a pulse waveform specified based on the pulse wave information, the event determination unit may determine that the subject is in the pulse missing state.

Therefore, for example, by adjusting the given period of time, it is possible to adjust the state of the subject determined to be in a pulse missing state.

In the present embodiment, when it is determined that the subject is in an atrial fibrillation state based on the pulse wave information, the event determination unit may determine that the syncope event has occurred in the subject.

Therefore, it is possible to store the pulse wave information when the subject is in an atrial fibrillation state or to send notification indicating that the subject is in an atrial fibrillation state, for example.

In the present embodiment, when it is determined that blood oxygen saturation of the subject is equal to or less than a given set value, the event determination unit may determine that the syncope event has occurred in the subject.

Therefore, it is possible to store the pulse wave information when the blood oxygen saturation (SpO₂) of the subject is equal to or less than the given set value or to send notification indicating that the subject is in a pulse missing state, for example.

In the present embodiment, when it is determined that the subject has fallen down due to syncope based on body motion sensor information obtained from a body motion sensor, the event determination unit may determine that the syncope event has occurred in the subject.

Therefore, it is possible to store the pulse wave information when the subject has fallen down due to syncope, for example.

In the present embodiment, the pulse wave information at the time of occurrence of the syncope event may include information of at least one of a pulse rate at the time of occurrence of the syncope event and a pulse waveform at the time of occurrence of the syncope event.

Therefore, it is possible to notify of at least one of the event pulse rate and the event pulse waveform using the notification unit.

In the present embodiment, the control unit may output the stored pulse wave information at the time of occurrence of the syncope event as notification information that is notified by a notification unit.

Therefore, the subject or the doctor can see the pulse wave information at the time of the occurrence of an event.

In the present embodiment, the control unit may calculate autonomic nervous activity information of the subject based on the pulse wave information, associate the autonomic nervous activity information with the pulse wave information at the time of occurrence of the syncope event, and output the autonomic nervous activity information as notification information that is notified by the notification unit.

Therefore, the doctor or the like can see the autonomic nervous activity information of the subject at the time of the occurrence of a syncope event.

In the present embodiment, the event determination unit may determine whether or not the syncope event has occurred based on reference pulse wave information for determination.

Therefore, it is possible to determine whether or not the currently acquired pulse wave information is abnormal compared with the reference pulse wave information.

In the present embodiment, the reference pulse wave information may be the pulse wave information corresponding to a timing of receiving an operation of a user.

Therefore, even if the reference pulse wave information is not registered in advance, it is possible to newly register reference pulse wave information whenever the subject performs measurement processing.

In the present embodiment, the event determination unit may determine whether or not the syncope event has occurred by comparing the reference pulse wave information with the pulse wave information measured by the pulse wave information measuring unit.

Therefore, when it can be determined that an abnormality, such as bradycardia or tachycardia, has occurred in the subject compared with the normal state, it is possible to determine that a syncope event has occurred.

In the present embodiment, syncope index information that changes according to a situation of a sign of syncope or development of syncope in the subject is calculated based on pulse wave amplitude information of a plurality of pulsations within a given period of time.

Therefore, it is possible to output it to the viewer, as more understandable information, that there has been a sign of syncope or syncope has been developed.

In the present embodiment, the control unit may calculate the syncope index information based on a total value of the pulse wave amplitude information of the plurality of pulsations within the given period of time.

Therefore, it is possible to calculate the syncope index information not only from the pulse wave information at a certain timing but also from the pulse wave information in a given period of time for which a syncope event has occurred.

In the present embodiment, the control unit may calculate the syncope index information by performing multiplication processing between the total value and a coefficient for calculation of the syncope index information.

Therefore, it is possible to adjust the rate of change of the syncope index information by adjusting the pulse coefficient.

In the present embodiment, the control unit may calculate the syncope index information by performing the multiplication processing between the total value and the coefficient that changes according to a pulse rate of the subject.

Therefore, it is possible to change the syncope index information according to the pulse rate.

In the present embodiment, the control unit may calculate the syncope index information by performing the multiplication processing between the total value and the coefficient that increases in a first pulse rate range and decreases in a second pulse rate range having the higher pulse rate than in the first pulse rate range.

Therefore, for example, when the subject is in a bradycardia state or in a tachycardia state, it is possible to reduce the value of the syncope index information.

In the present embodiment, the control unit may calculate the syncope index information by performing the multiplication processing between the total value and the coefficient that is a fixed value in a third pulse rate range between the first and second pulse rate ranges.

Therefore, for example, when the pulse rate of the subject is normal, it is possible to increase the value of the syncope index information.

In the present embodiment, the control unit may output the calculated syncope index information as notification information that is notified by a notification unit.

Therefore, since the notification unit can notify of the syncope index information, the doctor or the like can determine whether or not it is necessary to examine the state of the subject at that time in detail based on the syncope index information.

In the present embodiment, the control unit may output information obtained by associating the pulse wave information, from which the syncope index information has been calculated, with the syncope index information as notification information that is notified by the notification unit.

Therefore, for example, syncope index information and the pulse waveform corresponding to the syncope index information can be simultaneously displayed on the display unit.

In the present embodiment, the control unit may output the syncope index information at the time of occurrence of an event and the syncope index information in a normal state as the notification information that is notified by the notification unit.

Therefore, the doctor or the like can compare the syncope index information of the subject in the normal state with the syncope index information when a syncope event has occurred, and determine whether or not it is necessary to examine the state of the subject at that time in detail.

Hereinafter, the present embodiment will be described. In addition, the present embodiment described below is not intended to limit the contents of the invention defined by the appended claims. In addition, all components described below are not necessarily essential components of the invention.

### 1. Overview

The present embodiment relates to a biological information detecting device (syncope detecting device) using a wearable pulse wave measuring device that can be easily mounted. The wearable pulse wave measuring device is of a wristwatch type, for example. The biological information detecting device of the present embodiment includes a pulse wave sensor and other sensors mounted therein, and records a pulse wave in an internal memory while detecting a pulse wave at all times. Then, the biological information detecting device compares the pulse rate or the pulse wave amplitude value specified from the pulse wave with two values set in advance. When the pulse rate satisfies the given conditions, the biological information detecting device sends a warning to the subject and records the pulse wave at that time as an event pulse. Specifically, when the pulse rate becomes less than a first threshold value (example: less than 40 bpm), or when the pulse rate becomes a second threshold value or more (example: 200 bpm or more) so that the pulse wave amplitude starts to decrease, or when the pulse wave is missing for two seconds or more, the biological information detecting device sends a warning and records the pulse wave at that time as an event pulse. The event pulse is recorded up to the event pulse recording capacity set in advance.

Syncope due to arrhythmia may be caused by atrial fibrillation in addition to the cause of the above. Therefore, in the present embodiment, known atrial fibrillation determination processing is performed. When it is determined that the atrial fibrillation determination ratio exceeds the set value, a warning is sent as there is a possibility of syncope, and the pulse wave at that time is recorded as an event pulse.

It is also known that SpO₂ (blood oxygen saturation) is reduced when the subject faints. For this reason, SpO₂ detection processing is performed. When the SpO₂ value becomes equal to or less than a set value (for example, 80%), a warning is sent as there is a possibility of syncope, and the pulse wave at that time is recorded as an event pulse.

In the processing described above, a pulse wave may be transmitted to an electronic apparatus, such as a smartphone or a PC, using a wireless communication unit, and these may perform the recording. If the biological information detecting device and the electronic apparatus can communicate with each other, the electronic apparatus may send a warning.

When the subject faints, the subject may fall down. Therefore, when a multi-axis acceleration sensor provided in the wearable pulse wave measuring device exceeds a set value, the event is detected as a fall by syncope and a warning is sent, and the pulse wave for a few minutes before and after the event is recorded as an event pulse.

In the present embodiment, as described above, after recording the event pulse, the doctor or the like analyzes the recorded data. When analyzing the data, a biological information detecting device, a notification processing apparatus connected to the biological information detecting device by communication, or the like calculates an event pulse rate based on the pulse wave data of each event pulse that is stored. The event pulse rate is a pulse rate in a given pulse wave interval around the lowest pulse wave amplitude value in a series of pulse wave data recorded as an event pulse.

In this case, a biological information detecting device or the like calculates LF/HF as an index indicating the vagus nerve activity in the subject. In addition, a normal pulse rate is calculated from the pulse wave data other than the event pulse.

The event pulse wave, the event pulse rate, the vagus nerve activity, the pulse wave in a normal state, the normal pulse rate, and the like are displayed on a display unit.

From this display, the doctor determines whether the sign of syncope of the subject is due to arrhythmia (tachycardia or bradycardia) or due to vagus nerve. As described above, these analysis processing and display processing may be performed by an electronic apparatus (notification processing apparatus), such as a smartphone or a PC.

As described above, according to the present embodiment, unlike the related art in which an electrode is bonded onto the skin to detect the ECG or unlike an implantable electrocardiograph, it is possible to monitor the pulse wave of the subject in daily life for a long period of time. Therefore, it is possible to monitor or help to diagnose a patient with a syncope symptom.

### 2. Example of the System Configuration

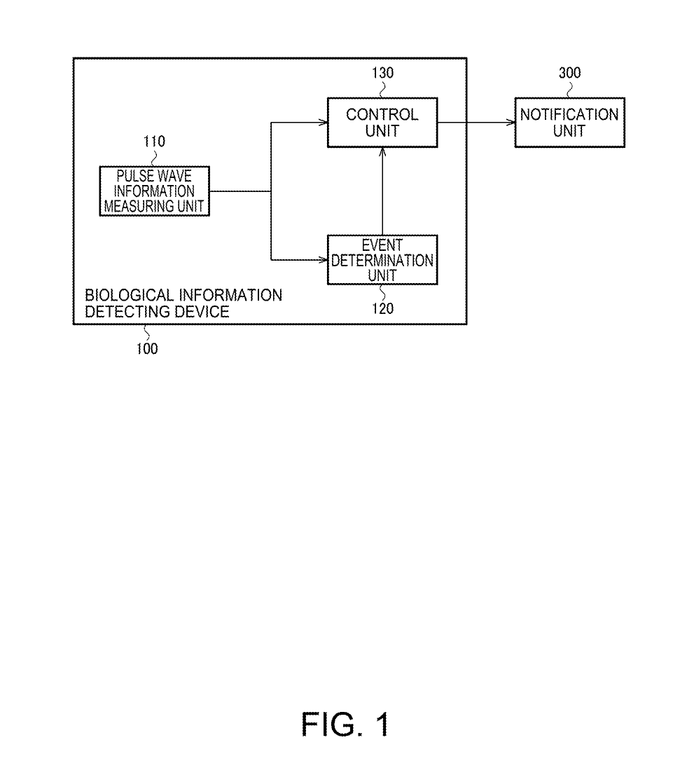

Next, FIG. 1 shows an example of the configuration of the biological information detecting device of the present embodiment.

A biological information detecting device 100 of the present embodiment includes a pulse wave information measuring unit 110, an event determination unit 120, and a control unit 130. The biological information detecting device 100 is not limited to the configuration shown in FIG. 1, and various modifications can be made. For example, some of the components may be omitted, or other components may be added. Some or all of the functions of the biological information detecting device 100 are realized by a wearable pulse wave measuring device, for example. In addition, some or all of the functions of the biological information detecting device 100 of the present embodiment may be realized by a portable electronic device or a server connected to the biological information detecting device 100 by communication.

Next, processing performed in each unit will be described.

The pulse wave information measuring unit 110 measures the pulse wave information of the subject. Here, the pulse wave information measuring unit 110 is a pulse wave sensor, for example. The pulse wave sensor is a sensor for detecting a pulse wave sensor signal (pulse wave information). For example, a photoelectric sensor can be considered. However, other sensors, such as a sensor using an ultrasonic wave, may also be used without being limited to the use of the photoelectric sensor as a pulse wave sensor. In addition, a plurality of types of sensors may be provided without being limited to the case in which only one sensor is provided as a pulse wave sensor. When a plurality of pulse wave sensors are provided, the respective sensors may be provided at different positions (parts) of the biological information detecting device 100.

Based on the measured pulse wave information, the event determination unit 120 determines whether or not a syncope event, such as the sign of syncope or the development of syncope, has occurred in the subject.

When it is determined that a syncope event has occurred, the control unit 130 performs at least one of storage processing of pulse wave information when the syncope event has occurred and syncope event notification processing. The functions of the event determination unit 120 and the control unit 130 can be realized by hardware, such as various processors (for example, a CPU) and an ASIC (for example, a gate array), a program, or the like.

That is, in the present embodiment, pulse wave information is measured, and it is determined whether or not a syncope event, such as the sign of syncope or the development of syncope, has occurred in the subject based on the measured pulse wave information. Then, when it is determined that a syncope event has occurred, at least one of the storage processing of the pulse wave information at the time of the occurrence of the syncope event and the syncope event notification processing is performed.

Therefore, for example, by storing the pulse wave information at the time of the occurrence of the syncope event, the doctor or the like can specify the cause of syncope by analyzing the pulse wave information at the time of the occurrence of the syncope event afterward. In addition, when a syncope event occurs, the subject is notified of the fact, so that the accident due to the development of syncope can be prevented.

The biological information detecting device of the present embodiment is a wristwatch type wearable pulse wave measuring device, for example. Therefore, for example, in order to measure the pulse wave using light or an ultrasonic wave, an electrocardiograph is not implanted into the body of the subject or the skin is not irritated unlike those described above. That is, even if the subject wears the biological information detecting device at all times, it is possible to suppress the physical burden on the subject.

As described above, according to the present embodiment, it is possible to detect the sign of syncope or the development of syncope while suppressing the physical burden on the subject.

### 3. Method of the Present Embodiment

Next, a method of the present embodiment will be described. First, a process of determining a syncope event will be described. In the present embodiment, as described above, it is determined that a syncope event has occurred when the subject is in a bradycardia state, or in a tachycardia state, or in a pulse missing state, for example. Hereinafter, each case will be described specifically.

First, the event determination unit 120 determines that a syncope event has occurred in the subject when it is determined that the subject is in a bradycardia state based on the pulse wave information.

Accordingly, it is possible to store the pulse wave information when the subject is in a bradycardia state or to send notification indicating that the subject is in a bradycardia state, for example.

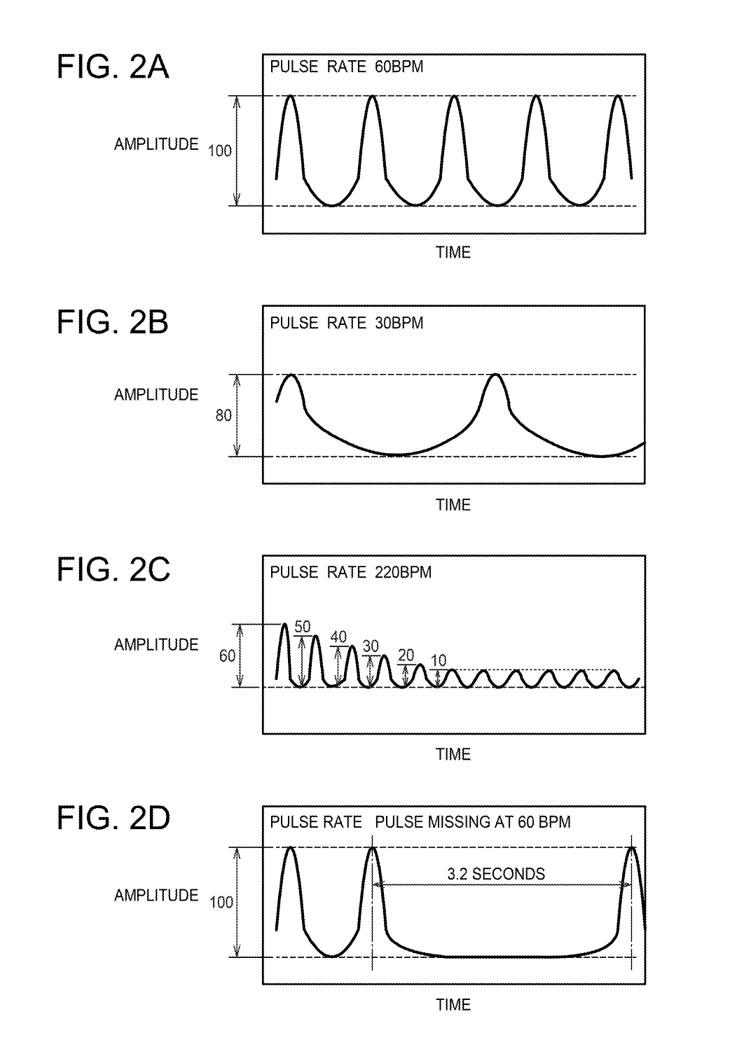

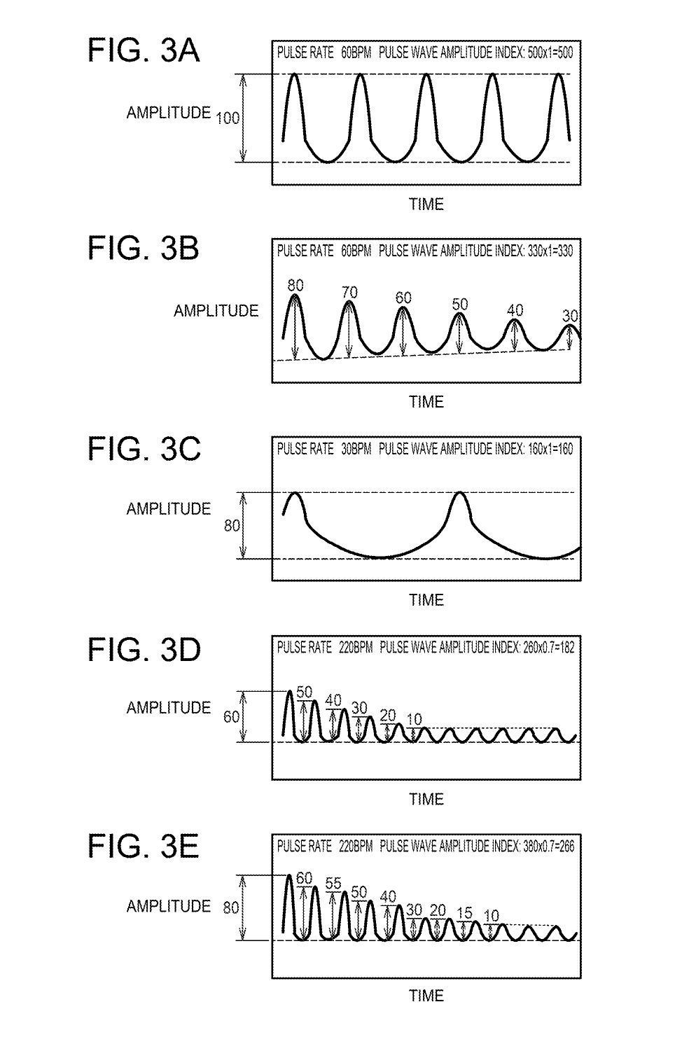

A specific example will be described. First, it is assumed that the pulse wave of the subject usually shows a waveform shown in FIG. 2A. In the graph shown in FIG. 2A, the vertical axis indicates amplitude, and the horizontal axis indicates a time. The same is true for the graphs shown in FIGS. 2B to 2D and 3A to 3E.

In the example shown in FIG. 2A, the pulse rate is 60 bpm, and the amplitude is 100. In the other graphs shown in FIGS. 2B to 2D and 3A to 3E, the amplitude is expressed as a relative value when the amplitude of the pulse waveform shown in FIG. 2A is 100.

The event determination unit 120 determines that the subject is in a bradycardia state when the pulse rate specified based on the pulse wave information is less than the first threshold value.

For example, in the example shown in FIG. 2B, the pulse rate is 30 bpm. Assuming that the first threshold value for determining that the subject is in a bradycardia state is, for example, 40 bpm, in the case shown in FIG. 2B, it can be determined that the subject is in a bradycardia state since the pulse rate is less than the first threshold value.

Therefore, by adjusting the first threshold value, it is possible to adjust the state of the subject determined to be in a bradycardia state.

In addition, the event determination unit 120 determines that a syncope event has occurred in the subject when it is determined that the subject is in a tachycardia state based on the pulse wave information.

Accordingly, it is possible to store the pulse wave information when the subject is in a tachycardia state or to send notification indicating that the subject is in a tachycardia state, for example.

Specifically, when the pulse rate specified based on the pulse wave information is equal to or greater than the second threshold value and the pulse wave amplitude at a first timing specified based on the pulse wave information is greater than the pulse wave amplitude at a second timing after the first timing, the event determination unit 120 determines that the subject is in a tachycardia state.

For example, in the example shown in FIG. 2C, the pulse rate is 220 bpm. Assuming that the second threshold value for determining that the subject is in a tachycardia state is, for example, 200 bpm, in the case shown in FIG. 2C, the pulse rate is equal to or greater than the second threshold value. In the example shown in FIG. 2C, the first amplitude of the pulse waveform stored as an event pulse is 60, but the amplitude decreases gradually as 50, 40, 30, 20, and 10. In this case, it can be determined that the subject is in a tachycardia state.

Therefore, by adjusting the second threshold value, it is possible to adjust the state of the subject determined to be in a tachycardia state.

In addition, the event determination unit 120 determines that a syncope event has occurred in the subject when it is determined that the subject is in a pulse missing state based on the pulse wave information.

Accordingly, it is possible to store the pulse wave information when the subject is in a pulse missing state or to send notification indicating that the subject is in a pulse missing state, for example.

Specifically, when the pulse rate specified based on the pulse wave information is equal to or greater than a third threshold value and less than a fourth threshold value and no pulsation is detected over a given period of time in the pulse waveform specified based on the pulse wave information, the event determination unit 120 determines that the subject is in a pulse missing state.

For example, in the example shown in FIG. 2D, the pulse rate is 60 bpm. Assuming that the third and fourth threshold values for determining that the subject is in a pulse missing state are 40 bpm and 200 bpm, respectively, in the case shown in FIG. 2C, the pulse rate is equal to or greater than the third threshold value and less than the fourth threshold value. The third threshold value may be the same value as the first threshold value, or may be a different value from the first threshold value. Similarly, the fourth threshold value may be the same value as the second threshold value, or may be a different value from the second threshold value. The fourth threshold value is a larger value than the third threshold value.

In the example shown in FIG. 2D, a period for which no pulsation can be detected is 3.2 seconds. Therefore, it can be determined that the subject is in a pulse missing state. Here, the given period of time for which no pulsation can be detected is set to 2 seconds or more.

Therefore, for example, by adjusting the given period of time, it is possible to adjust the state of the subject determined to be in a pulse missing state.

In the present embodiment, in addition to the above, for example, the event determination unit 120 determines that a syncope event has occurred in the subject when it is determined that the subject is in an atrial fibrillation state based on the pulse wave information. Determination regarding whether or not the subject is in an atrial fibrillation state is performed using a known method.

Accordingly, it is possible to store the pulse wave information when the subject is in an atrial fibrillation state or to send notification indicating that the subject is in an atrial fibrillation state, for example.

Also when it is determined that the blood oxygen saturation (SpO₂) of the subject is equal to or less than a given set value, the event determination unit 120 determines that a syncope event has occurred in the subject. For example, when the given set value is 80% and the blood oxygen saturation (SpO₂) is determined to be equal to or less than 80%, the event determination unit 120 determines that a syncope event has occurred in the subject.

Accordingly, it is possible to store the pulse wave information when the blood oxygen saturation (SpO₂) of the subject is equal to or less than the given set value or to send notification indicating that the subject is in a pulse missing state, for example.

The event determination unit 120 determines that a syncope event has occurred in the subject when it is determined that the subject has fallen down due to syncope based on the body motion sensor information obtained from a body motion sensor (not shown). In this case, a case is assumed in which, instead of the sign of syncope, syncope is developed and the subject falls down due to the syncope.

Accordingly, it is possible to store the pulse wave information when the subject has fallen down due to syncope, for example.

Then, the control unit 130 outputs the stored pulse wave information at the time of the occurrence of the syncope event as notification information that is notified by a notification unit 300.

Therefore, the subject or the doctor can see the pulse wave information at the time of the occurrence of an event.

Here, the pulse wave information at the time of the occurrence of a syncope event includes information of at least one of the pulse rate at the time of the occurrence of a syncope event (hereinafter, referred to as an event pulse rate) and the pulse waveform at the time of the occurrence of a syncope event (hereinafter, referred to as an event pulse waveform). However, the pulse wave information at the time of the occurrence of a syncope event is not limited to such information.

Thus, it is possible to notify of at least one of the event pulse rate and the event pulse waveform shown in FIGS. 2B to 2D using the notification unit 300.

In addition, the control unit 130 calculates the autonomic nervous activity information of the subject based on the pulse wave information, associates the autonomic nervous activity information with the pulse wave information at the time of the occurrence of a syncope event, and outputs the autonomic nervous activity information as notification information that is notified by the notification unit 300.

Therefore, the doctor or the like can see the autonomic nervous activity information of the subject at the time of the occurrence of a syncope event.

In addition, the event determination unit 120 determines whether or not a syncope event has occurred based on reference pulse wave information for determination.

For example, in the examples shown in FIGS. 2A to 2D described above, the reference pulse wave information is a given threshold value (40 bpm, 200 bpm, and the like) of the pulse rate determined in advance, a given pulse missing period (2 seconds or more), or the like.

Accordingly, it is possible to determine whether or not the currently acquired pulse wave information is abnormal compared with the reference pulse wave information.

In addition, the reference pulse wave information may be pulse wave information corresponding to the timing of receiving the operation of the user (subject). That is, it may be determined that the subject is in a normal state at the timing of receiving the operation of the user first, and the pulse wave information at that time may be set as the reference pulse wave information.

For example, in the examples shown in FIGS. 2A to 2D described above, the pulse wave information (pulse rate of 60 bpm, amplitude of 100, and the like) shown in FIG. 2A is reference pulse wave information.

Therefore, even if the reference pulse wave information is not registered in advance, it is possible to newly register reference pulse wave information whenever the subject performs measurement processing.

Then, the event determination unit 120 may determine whether or not a syncope event has occurred by comparing the reference pulse wave information with the pulse wave information measured by the pulse wave information measuring unit 110.

For example, in the example shown in FIG. 2B, it may be determined that the subject is in a bradycardia state by comparing the current pulse rate (30 bpm) with the reference pulse rate (40 bpm). In the example shown in FIG. 2C, it may be determined that the subject is in a tachycardia state by comparing the current pulse rate (220 bpm) with the pulse rate (20 bpm) of the reference pulse wave information and comparing the amplitude (50 to 10) with the pulse waveform amplitude (100) in FIG. 2A.

Thus, when it can be determined that an abnormality, such as bradycardia or tachycardia, has occurred in the subject compared with the normal state, it is possible to determine that a syncope event has occurred.

In addition, when the notification unit 300 notifies the subject or the doctor that a syncope event has occurred, it is desirable to present information that is easy to understand at a glance. Although the doctor or the like can also read the state relevant to the syncope of the subject from the pulse waveform, it is possible to determine the state relevant to the syncope of the subject more easily with an index, such as a numerical value. If rough determination regarding whether or not an abnormality has occurred in the subject that can be performed based on one numerical value, it is also possible to quickly determine whether or not it is better to examine the state of the subject at that time in more detail.

Therefore, in the present embodiment, the control unit 130 calculates syncope index information, which changes according to the situation of the sign of syncope or the development of syncope in the subject, based on the measured pulse wave information.

The syncope index information is information that is used when, for example, a doctor or the like determines whether or not there is a sign of syncope in the subject or syncope has been developed in the subject. For example, the syncope index information is a numerical value that changes according to the situation of the sign of syncope or the development of syncope in the subject. More specifically, in examples shown in FIGS. 3A to 3E, the syncope index information is a pulse wave amplitude index, for example.

The control unit 130 calculates the syncope index information based on the pulse wave amplitude information of a plurality of pulsations within a given period of time.

Here, the pulsation (pulse) refers to the beating of the artery in which the amplitude of the pulse wave is maximized. For example, in the example shown in FIG. 3A, the number of times in which the amplitude of the pulse wave is maximized is 5. Accordingly, five pulsations are included. The five pulsations correspond to a plurality of pulsations in the given period of time described above.

Specifically, the pulse wave amplitude information can be calculated by the following Equation (1). In the following Equation (1), xᵢ is the amplitude of each wave of the pulse waveform stored as an event pulse, and i is a number indicating each wave. In addition, a is a coefficient that changes according to the pulse rate of the subject, which will be described later.

I=aΣxᵢ (1)

For example, a pulse waveform in a normal state is shown in FIG. 3A. In this case, the pulse wave amplitude index of 500 is calculated by adding five waves having an amplitude of 100 and multiplying the addition result by the coefficient a=1.

FIG. 3B shows an example in which the pulse rate is 60 bpm as in FIG. 3A but the amplitude decreases gradually. In this case, the pulse wave amplitude index obtained by adding the amplitude of each wave and multiplying the addition result by the coefficient a=1 is 330. Since this value is obviously smaller than the value 500 in FIG. 3A, the doctor or the like can determine instantaneously that a certain abnormality has occurred.

FIG. 3C shows an example of the bradycardia state. In this case, the total value of the pulse rate is reduced to 160, and the pulse wave amplitude index of 160 is obtained by multiplying the total value by the coefficient a=1. Since this value is obviously smaller than the value 500 in FIG. 3A, the doctor or the like can determine instantaneously that a certain abnormality has occurred.

In summary, first, the control unit 130 calculates the syncope index information based on the pulse wave information corresponding to the timing determined that a syncope event has occurred.

Therefore, as will be described later, it is possible to calculate the syncope index information indicating the state of the subject at the time of the occurrence of a syncope event.

Then, the control unit 130 calculates the syncope index information based on the total value of the pulse wave amplitude information of a plurality of pulsations within a given period of time. For example, in the example shown in FIG. 3A, the given period of time is a period of five waves of the pulse waveform, and the plurality of pulsations are the five waves. The pulse wave amplitude information is the amplitude (100) of each of the five waves, and the total value is 500.

Therefore, it is possible to calculate the syncope index information not only from the pulse wave information at a certain timing but also from the pulse wave information in a given period of time for which a syncope event has occurred. As a result, it is possible to prevent the erroneous determination of the doctor or the like by reducing the influence of noise included in the pulse wave information.

In addition, the control unit 130 calculates the syncope index information by performing multiplication processing between the total value of the pulse wave amplitude information of a plurality of pulsations within a given period of time and a coefficient for the calculation of syncope index information.

This coefficient (hereinafter, referred to as a pulse coefficient) is a numerical value that changes according to the pulse rate of the subject. In other words, the control unit 130 calculates the syncope index information by performing multiplication processing between the coefficient that changes according to the pulse rate of the subject and the total value of the pulse wave amplitude information described above.

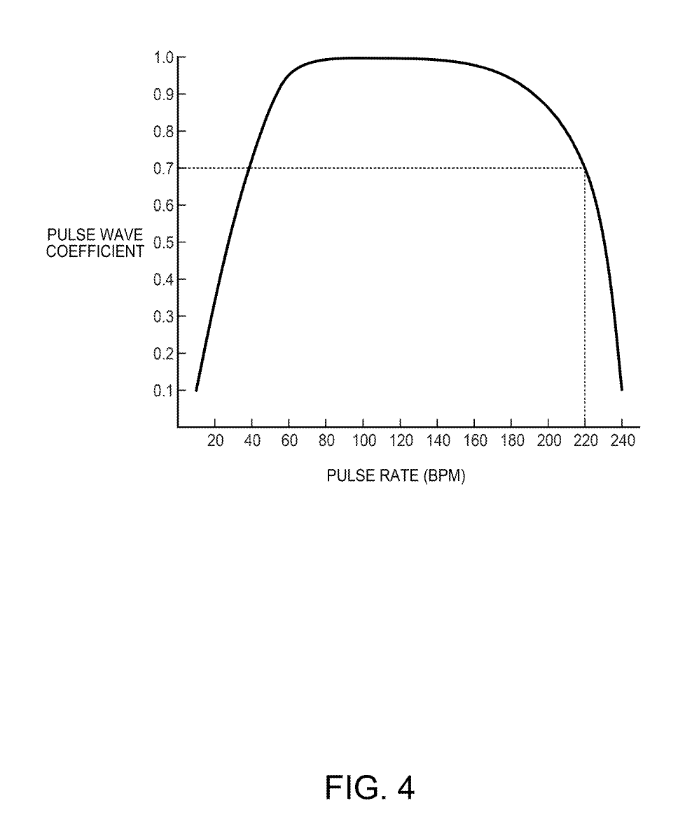

Specifically, the pulse coefficient is a coefficient shown in the graph of FIG. 4, for example. In the graph of FIG. 4, the vertical axis indicates a pulse coefficient, and the horizontal axis indicates a pulse rate (bpm).

For example, in the case shown in FIG. 3D, the pulse rate is 220 bpm, and the subject is in a tachycardia state. When the pulse rate is 220 bpm, the pulse coefficient of 0.7 is calculated from the graph shown in FIG. 4. Therefore, the pulse wave amplitude index of 182 is obtained by multiplying the total value 260 of the pulse rate in the example shown in FIG. 3D by the coefficient a=0.7. Since this value is obviously smaller than the value 500 in FIG. 3A, the doctor or the like can determine instantaneously that a certain abnormality has occurred.

Also in the case shown in FIG. 3E, similar to the case shown in FIG. 3D, since the subject is in a tachycardia state, the pulse wave amplitude index is 266. Therefore, it is possible to determine that an abnormality has occurred.

In this manner, it is possible to adjust the rate of change of the syncope index information by adjusting the pulse coefficient.

That is, as will be described in detail below, it is possible to change the syncope index information according to the pulse rate.

The pulse coefficient increases in a first pulse rate range, and decreases in a second pulse rate range having a higher pulse rate than in the first pulse rate range. That is, the control unit 130 calculates the syncope index information by performing multiplication processing between the coefficient, which increases in the first pulse rate range and decreases in the second pulse rate range having a higher pulse rate than in the first pulse rate range, and the total value.

In the example shown in FIG. 4, a range of approximately 40 bpm or less is the first pulse rate range, and a range of approximately 140 bpm or more is the second pulse rate range.

Therefore, when the subject is in a bradycardia state or in a tachycardia state, it is possible to reduce the value of the syncope index information.

In addition, the pulse coefficient is a fixed value in a third pulse rate range between the first and second pulse rate ranges. That is, the control unit 130 calculates the syncope index information by performing multiplication processing between the coefficient, which is a fixed value in the third pulse rate range between the first and second pulse rate ranges, and the total value.

In the example shown in FIG. 4, a range of approximately 40 bpm or more and less than approximately 140 bpm is the third pulse rate range.

Therefore, when the pulse rate of the subject is normal, it is possible to increase the value of the syncope index information. In addition, the pulse coefficient may be a value based on the pulse wave propagation speed or may be a value based on the degree of arteriosclerosis, for example. Here, the pulse wave propagation speed is calculated based on a period from the timing at which the R wave is detected in the electrocardiogram to the timing at which the pulse wave is detected by the biological information detecting device. In this case, the biological information detecting device having an electrocardiograph and having an electrode serving as aback cover is worn on the left wrist of the subject. Then, the subject touches the bezel (electrode) of the biological information detecting device with the right hand, so that the electrocardiograph in the biological information detecting device detects and records the induced electrocardiogram. Then, as described above, the biological information detecting device calculates the pulse wave propagation speed based on the detected electrocardiogram. Through the configuration described above, it is possible to set the pulse coefficient by combining the information of the pulse wave and the electrocardiogram. Therefore, it is possible to set a more accurate value.

In addition, the control unit 130 outputs the calculated syncope index information as notification information that is notified by the notification unit 300.

Therefore, since the notification unit 300 can notify of the syncope index information, the doctor or the like can determine whether or not it is necessary to examine the state of the subject at that time in detail based on the syncope index information.

In addition, the control unit 130 outputs information obtained by associating the pulse wave information, from which syncope index information is calculated, with the syncope index information as notification information that is notified by the notification unit 300.

Therefore, for example, syncope index information and the pulse waveform corresponding to the syncope index information can be simultaneously displayed on the display unit. As a result, when the doctor or the like determines that there is an abnormality when watching the syncope index information and that detailed examination is required, it is possible to see the pulse waveform at that time immediately.

In addition, the control unit 130 outputs syncope index information at the time of the occurrence of an event and syncope index information in the normal state as notification information that is notified by the notification unit 300.

Therefore, the doctor or the like can compare the syncope index information of the subject in the normal state with the syncope index information when a syncope event has occurred, and determine whether or not it is necessary to examine the state of the subject at that time in detail.

In this manner, it is possible to present it to the viewer, as more understandable information, that there has been a sign of syncope or syncope has been developed.

### 4. Details of Processing

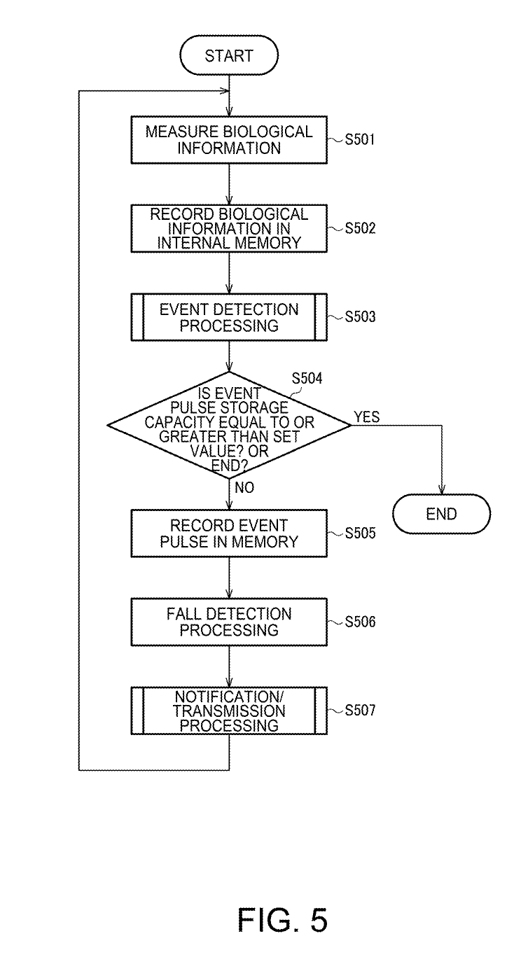

Next, the rough flow of the process of the present embodiment will be described with reference to the flowchart shown in FIG. 5.

First, the pulse wave information measuring unit 110 measures biological information (pulse wave information) (S501), and stores the measured biological information in the internal memory (not shown) (S502). Then, the event determination unit 120 performs event detection processing for a syncope event based on the measured biological information (S503). Details of the event detection processing will be described later.

Then, the control unit 130 determines whether or not the storage capacity of the pulse wave information of the event pulse stored in the internal memory is equal to or greater than the set value or whether or not the biological information measurement processing has ended (S504).

Then, when it is determined that the storage capacity of the pulse wave information of the event pulse is less than the set value and the measurement processing has not ended, the control unit 130 stores the pulse wave information of the event pulse, which has been newly detected in step S503, in the internal memory (S505).

Then, the control unit 130 performs the fall detection processing described above (S506).

Then, when a syncope event has occurred, the control unit 130 performs at least one of the syncope event notification processing using the notification unit 300 and processing of transmitting the pulse wave information of the syncope event to an external device (S507), and the process returns to step S501. The specific flow of the notification processing will be described in detail later.

When it is determined that the storage capacity of the pulse wave information of the event pulse is equal to or greater than the set value or when it is determined that the measurement processing has ended in step S504, the control unit 130 ends the process.

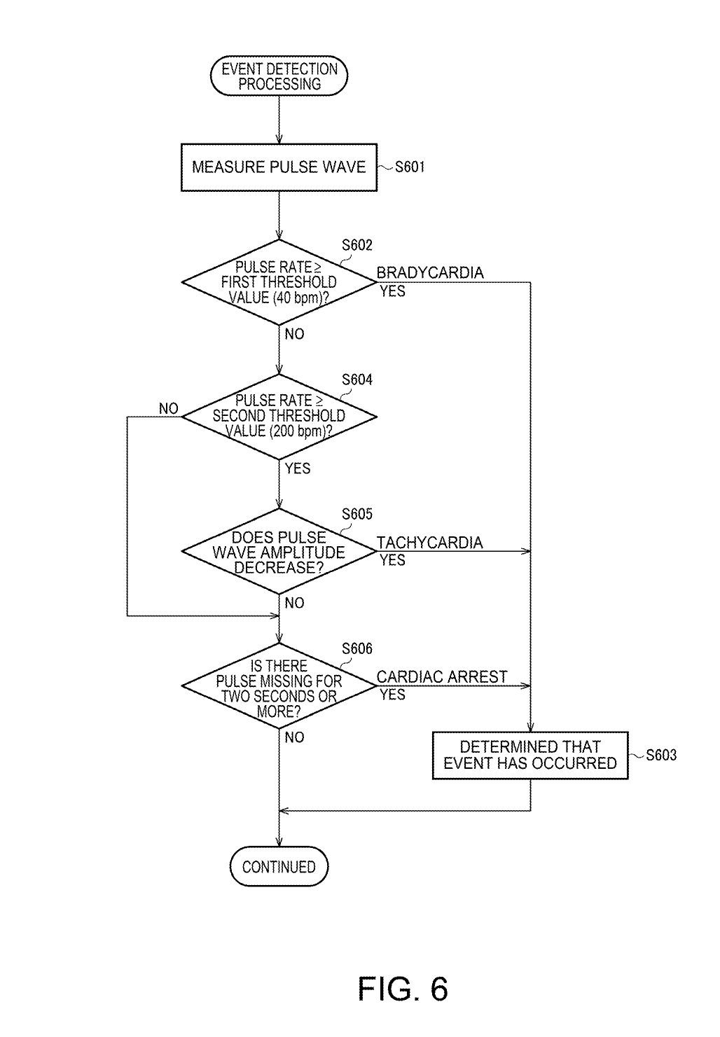

Next, details of the event detection processing will be described with reference to the flowchart shown in FIG. 6.

First, the pulse wave information measuring unit 110 specifies pulse wave information from the biological information detected in step S501 of FIG. 5 (S601). The biological information may include various kinds of measurement data regarding the body of the subject in addition to the pulse wave information.

Then, the event determination unit 120 specifies a pulse rate from the pulse wave information, and determines whether or not the specified pulse rate is equal to or less than the first threshold value (40 bpm) (S602).

When it is determined that the pulse rate is equal to or less than the first threshold value (40 bpm), the event determination unit 120 determines that the subject is in a bradycardia state (S603), and ends the event detection processing. For example, this corresponds to the above case shown in FIG. 2B.

On the other hand, when it is determined that the pulse rate is greater than the first threshold value (40 bpm), the event determination unit 120 determines that the subject is not in a bradycardia state. Then, it is determined whether or not the pulse rate is equal to or greater than the second threshold value (200 bpm) (S604).

When it is determined that the pulse rate is equal to or greater than the second threshold value (200 bpm), the event determination unit 120 further determines whether or not the pulse wave amplitude decreases gradually (S605). When it is determined that the pulse wave amplitude decreases gradually, the event determination unit 120 determines that the subject is in a tachycardia state (S603), and ends the event detection processing. For example, this corresponds to the above case shown in FIG. 2C.

On the other hand, when it is determined that the pulse rate is less than the second threshold value or when it is determined that the pulse rate is equal to or greater than the second threshold value but the pulse wave amplitude is not decreased, the event determination unit 120 determines that the subject is not in a tachycardia state. In this case, the event determination unit 120 determines whether or not there is pulse missing for two seconds or more (S606).

When it is determined that there is pulse missing for two seconds or more, the event determination unit 120 determines that the subject is in a pulse missing state (S603), and ends the event detection processing. For example, this corresponds to the above case shown in FIG. 2D.

On the other hand, when it is determined that there is no pulse missing for two seconds or more, the event determination unit 120 determines that a syncope event has not occurred since the subject is not in the pulse missing state, and ends the event detection processing.

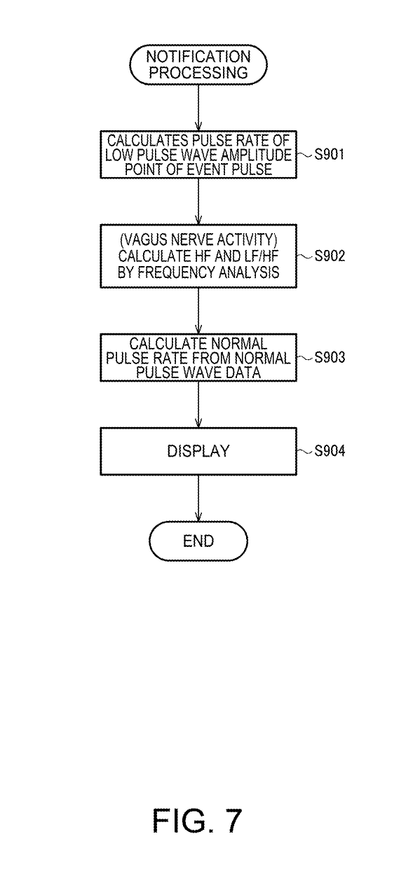

Next, the detailed flow of the notification processing in S507 of FIG. 5 will be described with reference to the flowchart shown in FIG. 7. A series of processes shown in FIG. 7 is performed, for example, when the analysis button of the biological information detecting device 100 is pressed by the user.

First, the control unit 130 calculates the pulse rate of the low pulse wave amplitude point of the event pulse (S901). Then, the control unit 130 calculates a high frequency (HF) and a low frequency (LF)/HF, as an index indicating the vagus nerve activity of the subject, by frequency analysis (S902). Then, the control unit 130 calculates the normal pulse rate from the normal pulse wave data (S903), and displays these data on the display unit (notification unit 300) and ends the processing. These processes may be performed by the biological information detecting device 100, or may be performed by an external information processing apparatus.

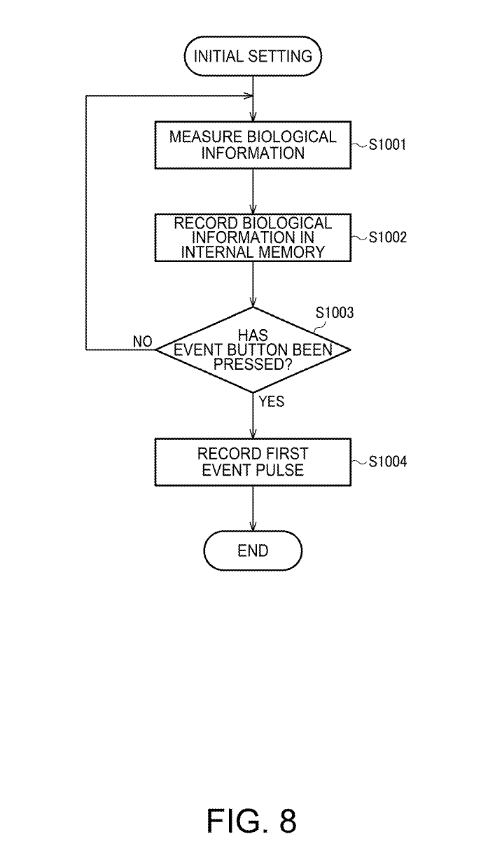

The flow of the process when calculating the syncope index information is shown in the flowcharts of FIGS. 8 to 12.

In this case, it is necessary to perform an initial setting first. In the initial setting, as shown in the flowchart of FIG. 8, first, the pulse wave information measuring unit 110 measures biological information (pulse wave information) (S1001), and stores the measured biological information in the internal memory (not shown) (S1002). Then, the control unit 130 determines whether or not the event button provided in the biological information detecting device 100 has been pressed (S1003).

In this example, for example, when the subject feels a hypotensive state (cerebral ischemia symptom), such as dizziness, or fatigue (cardiac output reduction) or breathlessness during exertion, the subject presses the event button. When it is determined that the event button provided in the biological information detecting device 100 has been pressed, the control unit 130 records the time when the event button has been pressed and the pulse wave for several minutes before and after the time as a first event pulse (S1004), and ends the initial setting.

On the other hand, when it is determined that the event button provided in the biological information detecting device 100 has been pressed, the control unit 130 returns to step S1001 and repeats the process until the first event pulse is recorded.

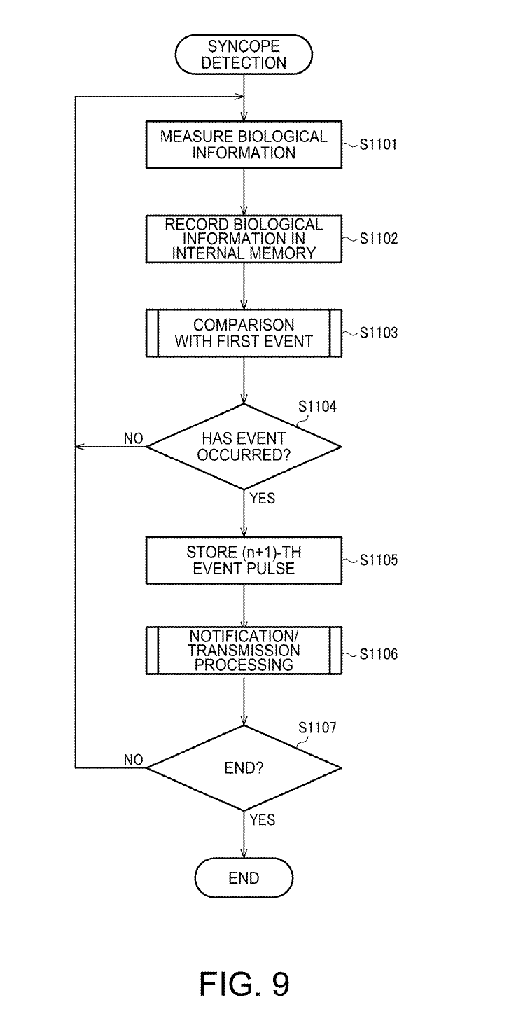

Next, the flow of the faint detection processing will be described with reference to the flowchart shown in FIG. 9. First, similar to steps S1001 and S1002, the pulse wave information measuring unit 110 measures biological information (pulse wave information) (S1101), and stores the measured biological information in the internal memory (not shown) (S1102).

Then, the event determination unit 120 performs comparison with the first event pulse (S1103). The detailed flow of this process will be described later.

Then, the event determination unit 120 determines whether or not a syncope event has occurred based on the comparison result in step S1103 (S1104). When it is determined that a syncope event has occurred, the pulse wave information of the currently measured pulse is stored in the internal memory as pulse wave information of the (n+1)-th event pulse (S1105). n is an integer of 1 or more. For example, the pulse waveform of the (n+1)-th event pulse is a pulse waveform shown in FIG. 3B or 3E described above.

Then, the control unit 130 performs at least one of the syncope event notification processing using the notification unit 300 and processing of transmitting the pulse wave information of the syncope event to an external device (S1106). Then, when there is an end instruction, the process is ended. On the other hand, when there is no end instruction, the process returns to step S1101. The specific flow of the notification processing will be described in detail later.

In addition, also when it is determined that no syncope event has occurred in step S1104, the process returns to step S1101 and repeats the process.

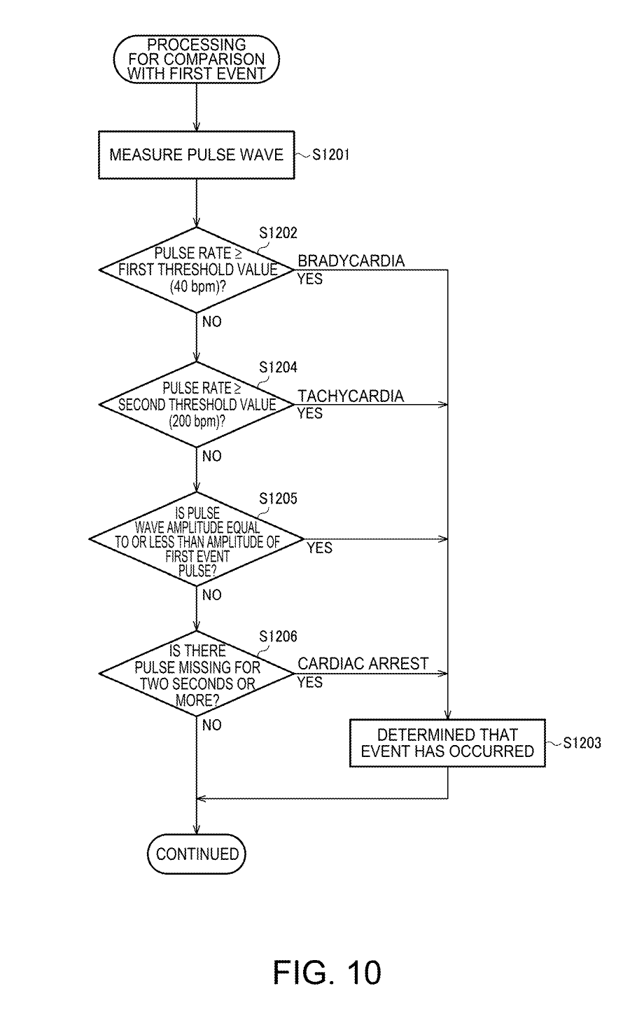

Next, the flow of the process for comparison with the first event pulse will be described with reference to the flowchart shown in FIG. 10. Since the processing other than step S1205 is the same as that in steps S601 to S604 and S606 shown in FIG. 6, the explanation thereof will be omitted.

In the step S605 shown in FIG. 6, the control unit 130 determines whether or not the pulse wave amplitude decreases gradually. However, in step S1205 shown in FIG. 10, the control unit 130 determines whether or not the currently measured pulse wave amplitude is equal to or less than the amplitude of the first event pulse.

When it is determined that the currently measured pulse wave amplitude is equal to or less than the amplitude of the first event pulse, the control unit 130 determines that a syncope event has occurred (S1203), and ends the process. That is, in the present embodiment, when it is determined that the sign of syncope or the degree of syncope indicated by the current pulse wave information is larger than that indicated by the pulse wave information of the first event pulse, the current pulse wave information is stored as the (n+1)-th event pulse (S1105).

On the other hand, when it is determined that the currently measured pulse wave amplitude is larger than the amplitude of the first event pulse, pulse missing determination processing in step S1206 is performed.

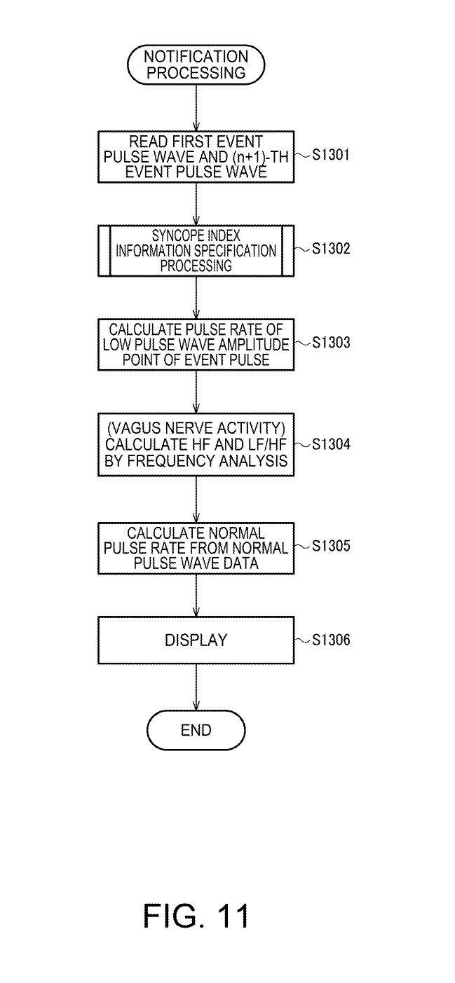

Next, the detailed flow of the notification processing in step S1106 shown in FIG. 9 will be described with reference to FIG. 11.

First, the control unit 130 reads the pulse wave information of the first event pulse and the pulse wave information of the (n+1)-th event pulse (S1301). Then, processing for specifying the syncope index information is performed (S1302). The detailed flow of the syncope index information specification processing will be described later. Since the process flow from step S1303 shown in FIG. 11 is the same as the process flow from step S901 shown in FIG. 7, the explanation thereof will be omitted.

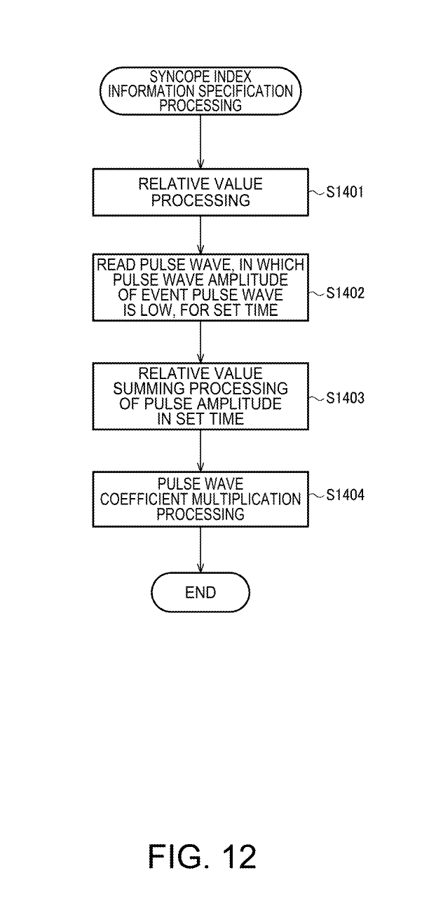

Next, the detailed flow of the syncope index information specification processing in step S1302 shown in FIG. 11 will be described with reference to FIG. 12.

First, the control unit 130 performs relative value processing for setting the pulse wave amplitude of the first event pulse to 100 for the pulse wave information of the first event pulse and the pulse wave information of the (n+1)-th event pulse acquired in step S1301 shown in FIG. 11 (S1401). Then, the control unit 130 reads a pulse wave in a period, in which the pulse wave amplitude of the event pulse wave is lower than a given threshold value, for a given set time (S1402).

In addition, the control unit 130 performs relative value summing processing of the pulse amplitude in the set time (S1403).

Finally, the control unit 130 performs pulse wave coefficient multiplication processing (S1404), and ends the process. That is, in steps S1403 and S1404, the processing of Equation (1) described above is performed.

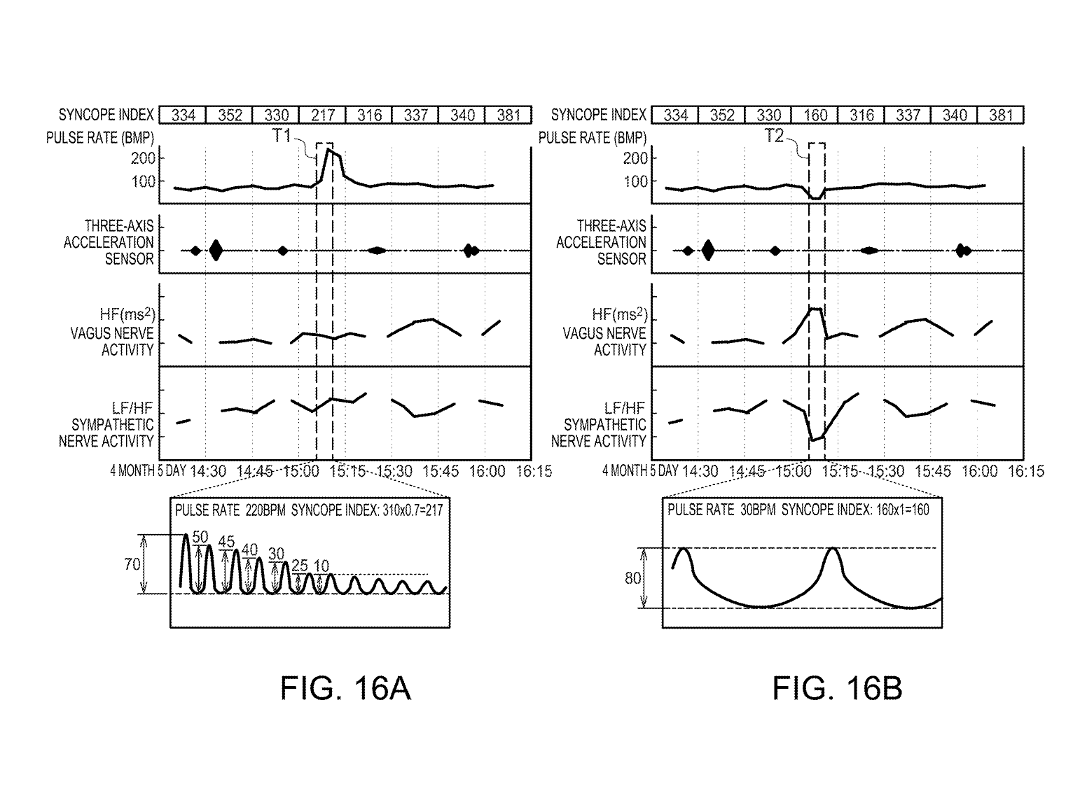

In step S1306 shown in FIG. 11 described above, display images shown in FIGS. 16A and 16B are displayed on the display unit (notification unit 300) based on the pulse wave information or the syncope index information calculated as described above. FIG. 16A shows an example of a display image when a syncope event due to tachycardia has occurred, and FIG. 16B shows an example of a display image when a syncope event due to bradycardia has occurred. In the present embodiment, the doctor or the like views the display images and analyzes the cause of syncope of the subject.

For example, the display image shown in FIG. 16A will be described in detail. In the display image in this example, syncope index information, a pulse rate (bpm), a sensor signal of a three-axis acceleration sensor, HF (ms²) indicating the vagus nerve activity, and LF/HF indicating the sympathetic nerve activity are displayed with the horizontal axis as a common time axis.

In the example shown in FIG. 16A, it can be seen that something abnormal has happened during the period of time of 15:00 to 15:15 since the syncope index information, which has been stable around 330 to 360, has greatly changed to 217. Therefore, by observing the other information in detail, it can be seen that the pulse rate has increased abruptly at the timing of T1. In addition, it can also be seen that there is no large change in the sensor signal of the acceleration sensor or the HF but the LF/HF has greatly changed. The more detailed analysis of the pulse rate in this case shows that the pulse rate increases but the amplitude of the pulse decreases as shown in the lower diagram of FIG. 16A. Therefore, it can be determined that the subject is in a tachycardia state. Thus, the doctor or the like can determine that the subject may fall into the tachycardia state and this can be the sign of syncope. In addition, the detailed image of the pulse rate shown in the lower diagram of FIG. 16A or the like may be appropriately displayed on the display unit in response to an instruction from the user.

Similarly, in the example shown in FIG. 16B, the syncope index information is greatly changed during the period of time of 15:00 to 15:15. By observing the information in this period in detail, it can be seen that the subject is in a bradycardia state since the pulse rate decreases and the HF or the LF/HF is greatly changed. Thus, the doctor or the like can determine that the subject may fall into the bradycardia state and this can be the sign of syncope.

### 5. An Example of the Configuration of the Biological Information Detecting Device

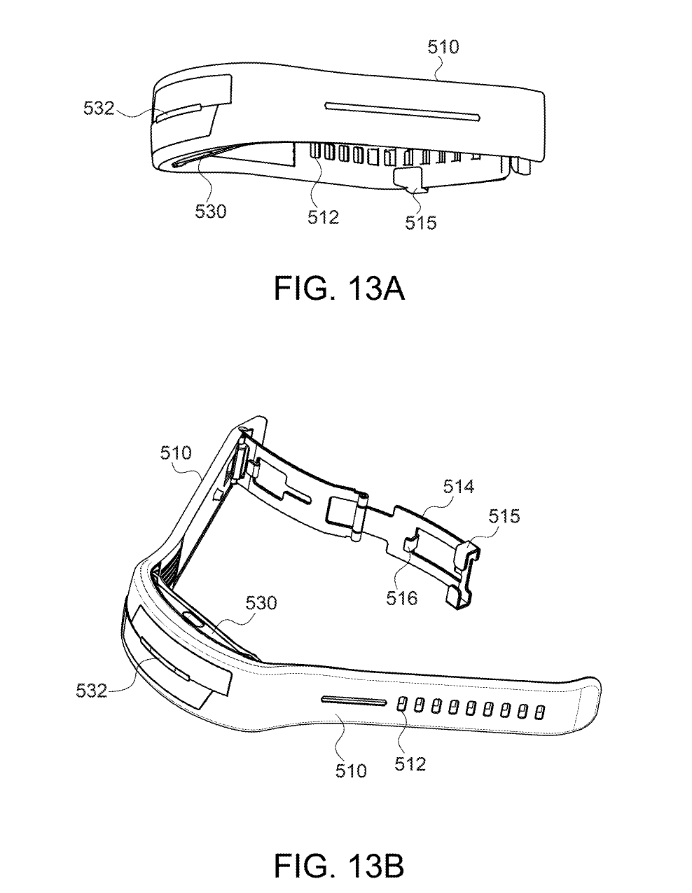

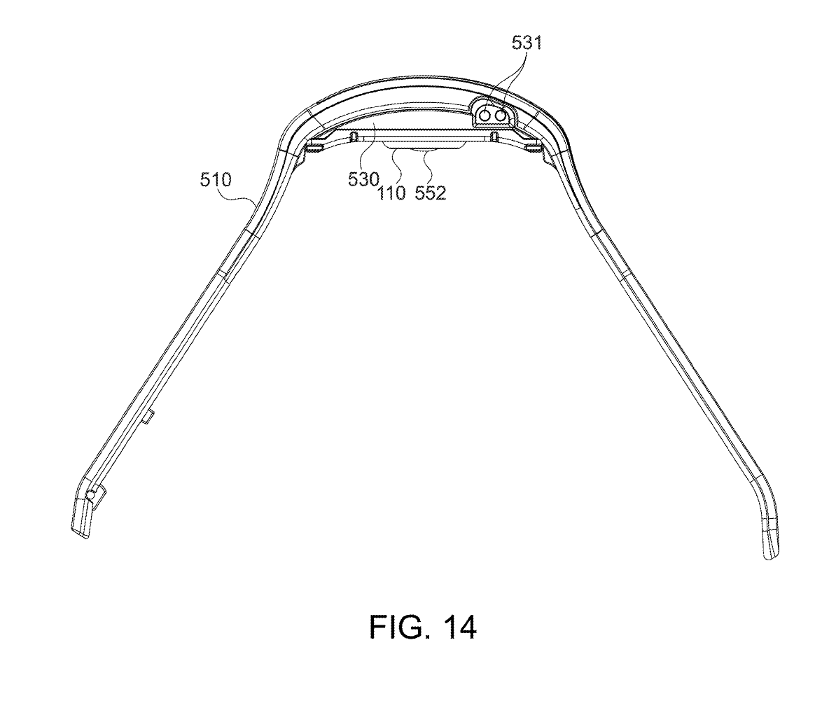

FIGS. 13A, 13B, and 14 show external views of the biological information detecting device of the present embodiment. FIG. 13A is a diagram when the biological information detecting device is viewed from the front direction side, FIG. 13B is a diagram when the biological information detecting device is viewed from above, and FIG. 14 is a diagram when the biological information detecting device is viewed from the side direction side.

As shown in FIGS. 13A, 13B, and 14, the biological information detecting device of the present embodiment includes a band unit 510, a case unit 530, and a sensor unit (pulse wave information measuring unit) 110. The case unit 530 is attached to the band unit 510. The sensor unit 110 is provided in the case unit 530. As shown in FIG. 1 described above, the biological information detecting device includes the event determination unit 120 and the control unit 130, and these are provided in the case unit 530. The biological information detecting device of the present embodiment is not limited to the configuration shown in FIGS. 13A, 13B, and 14, and various modifications can be made. For example, some of the components may be omitted or may be replaced with other components, or other components may be added.

The band unit 510 is wound around the user's wrist so that the biological information detecting device is mounted thereon. The band unit 510 includes a hole 512 and a buckle portion 514. The buckle portion 514 includes a band insertion portion 515 and a protruding portion 516. The user can wear the biological information detecting device on the wrist by inserting one end of the band unit 510 into the band insertion portion 515 of the buckle portion 514 and inserting the protruding portion 516 of the buckle portion 514 into the hole 512 of the band unit 510. In this case, depending on to which hole 512 the protruding portion 516 is inserted, the magnitude of the pressure on the sensor unit 110 (pressure against the wrist surface) is adjusted.

The case unit 530 corresponds to the main body of the biological information detecting device. Various components of the biological information detecting device, such as the sensor unit 110, the event determination unit 120, and the control unit 130, are provided in the case unit 530. That is, the case unit 530 is a housing in which these components are housed.

A light emitting window 532 is provided in the case unit 530. The light emitting window 532 is formed by a light-transmissive member. In addition, a light emitting portion (LED) mounted on a flexible substrate is provided in the case unit 530, and light from the light emitting portion is emitted to the outside of the case unit 530 through the light emitting window 532.

As shown in FIG. 14, a terminal portion 531 is provided in the case unit 530. When the biological information detecting device is mounted on a cradle (not shown), a terminal portion of the cradle and the terminal portion 531 of the case unit 530 are electrically connected to each other. Therefore, a secondary battery (battery) provided in the case unit 530 can be charged.

The sensor unit (pulse wave information measuring unit) 110 detects biological information, such as the pulse wave of the subject. For example, the sensor unit 110 includes a light receiving portion and a light emitting portion. Additionally, the sensor unit 110 is formed by a light-transmissive member, and includes a protruding portion 552 that applies pressure to the skin surface of the subject in contact with the skin surface. In a state in which the protruding portion 552 applies pressure to the skin surface as described above, the light emitting portion emits light, the light receiving portion receives light reflected by the subject (blood vessel), and the light receiving result is output to the control unit 130 as a detection signal. Then, the control unit 130 detects biological information, such as a pulse wave, based on the detection signal from the sensor unit 110. In addition, biological information to be detected by the biological information detecting device of the present embodiment is not limited to the pulse wave (pulse rate), and the biological information detecting device may detect other biological information (for example, blood oxygen saturation, body temperature, or a heart rate) that is not the pulse wave.

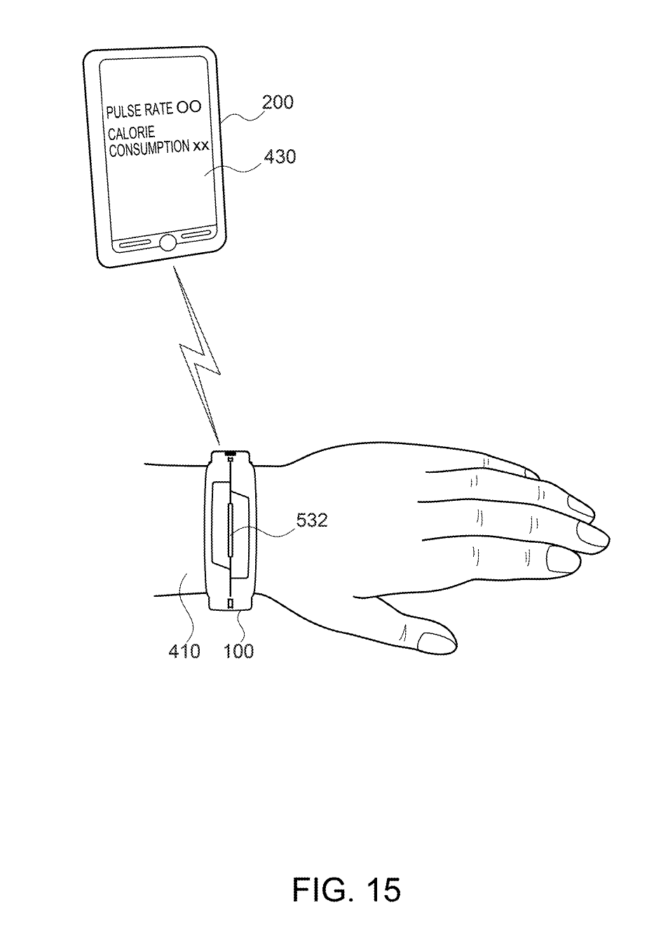

FIG. 15 is a diagram for explaining the mounting of the biological information detecting device 100 and communication with an information processing apparatus 200.

As shown in FIG. 15, a user who is a subject wears the biological information detecting device 100 on a wrist 410 as a watch. As shown in FIG. 14, the sensor unit 110 is provided on the surface of the case unit 530 on the subject side. When the biological information detecting device 100 is mounted, the protruding portion 552 of the sensor unit 110 applies pressure to the skin surface of the wrist 410 in contact with the skin surface, the light emitting portion of the sensor unit 110 emits light in the state, and the light receiving portion receives the reflected light. Accordingly, biological information, such as a pulse wave, is detected.

The biological information detecting device 100 and the information processing apparatus 200 are communicably connected to each other, so that the transmission and reception of data therebetween are possible. For example, the information processing apparatus 200 is a portable communication terminal, such as a smartphone, a mobile phone, and a future phone. Alternatively, the information processing apparatus 200 may be an information processing terminal, such as a tablet computer. As a communication connection between the biological information detecting device 100 and the information processing apparatus 200, for example, near field communication, such as Bluetooth, can be adopted. Thus, since the biological information detecting device 100 and the information processing apparatus 200 are communicably connected to each other, various kinds of biological information (first biological information and second biological information), such as a pulse rate or calorie consumption, can be displayed on a display unit 430 (LCD or the like) of the information processing apparatus 200. That is, various kinds of information calculated based on the detection signal of the sensor unit 110 can be displayed. The display unit 430 is an example of the notification unit 300 described above. In addition, arithmetic processing of the syncope index information or the like may be performed in the biological information detecting device 100, or at least apart of the arithmetic processing may be performed in the information processing apparatus 200.

A part or most of the processing of the biological information detecting device 100 of the present embodiment may be realized by a program. In this case, the biological information detecting device 100 of the present embodiment is realized when a processor, such as a CPU, executes a program. Specifically, a program stored in a non-transitory information storage device is read, and the read program is executed by a processor, such as a CPU. Here, the information storage device (device readable by the computer) is for storing a program, data, and the like, and its function can be realized by an optical disc (DVD, CD, or the like), a hard disk drive (HDD), a memory (card type memory, ROM, or the like), and the like. In addition, the processor, such as a CPU, performs various kinds of processing of the present embodiment based on the program (data) stored in the information storage device. That is, a program causing a computer (apparatus including an operation unit, a processing unit, a storage unit, and an output unit) to function as each unit of the present embodiment (program causing a computer to execute processing of each unit) is stored in the information storage device.

Therefore, it is possible to realize the processing of the present embodiment with a program. The program may be, for example, a program executed by being read into a processing unit (for example, a DSP) of a device, such as a smartphone.

The biological information detecting device 100 of the present embodiment may include a processor and a memory. The processor herein may be a central processing unit (CPU), for example. However, the processor is not limited to the CPU, and it is possible to use various processors, such as a graphics processing unit (GPU) or a digital signal processor (DSP). In addition, the processor may be a hardware circuit based on an application specific integrated circuit (ASIC). The memory is for storing a command that is readable by the computer. When the command is executed by the processor, each unit of the biological information detecting device 100 according to the present embodiment is realized. The memory herein may be a semiconductor memory, such as a static random access memory (SRAM) or a dynamic random access memory (DRAM), or may be a register, a hard disk, or the like. The command herein may be a command of a command set that forms a program, or may be a command to give an operation instruction to the hardware circuit of the processor.

In addition, a pulse wave detecting device that the user wears may be configured to include a pulse wave sensor 11 and a communication unit that communicates a pulse wave sensor signal from the pulse wave sensor 11 wirelessly or by cable. In this case, the program of the present embodiment is provided separately from the pulse wave detecting device, and is executed by being read from the above communication unit into a processing unit (for example, a CPU) of an information processing system that receives a pulse wave sensor signal. The information processing system may be one that is not worn by the user, such as a PC, or may be one that is worn (carried) by the user, such as a smartphone. A server system that is connected through a network, such as the Internet, may be used as the information processing system.

When the pulse wave detecting device and the information processing system in which a program is executed are separate bodies, a display unit that is used to present pulsation information to the user is provided in an arbitrary place. For example, the pulsation information may be displayed on the display unit of the information processing system, or a display unit may be provided in the pulse wave detecting device so that the pulsation information output from the information processing system is displayed thereon. Alternatively, the pulsation information may be displayed on the display unit of a different device (for example, an arbitrary client device when a server system is used as an information processing system).

While the present embodiment has been described in detail, it could be easily understood by those skilled in the art that various changes and modifications thereof could be made without departing from novel matters and effects of the invention. Therefore, such modifications are intended to be included within the scope of the invention. For example, in this specification or the diagrams, a term that is described at least once together with different terms having a broader meaning or the same meaning can be replaced with the different terms in any parts of the specification or diagrams. In addition, the configuration and operation of the biological information detecting device are not limited to those described in the present embodiment either, and modifications can be made.

What is claimed is:

1. A biological information detecting device, comprising:
a pulse wave information measuring unit that measures pulse wave information of a subject;
an event determination unit that determines whether or not a syncope event including a sign of syncope and development of syncope has occurred in the subject based on the measured pulse wave information; and
a control unit that performs at least one of storage processing of the pulse wave information at the time of occurrence of the syncope event and notification processing of the syncope event when it is determined that the syncope event has occurred.

2. The biological information detecting device according to claim 1,
wherein, when it is determined that the subject is in a bradycardia state based on the pulse wave information, the event determination unit determines that the syncope event has occurred in the subject.

3. The biological information detecting device according to claim 2,
wherein, when a pulse rate specified based on the pulse wave information is less than a first threshold value, the event determination unit determines that the subject is in a bradycardia state.

4. The biological information detecting device according to claim 1,
wherein, when it is determined that the subject is in a tachycardia state based on the pulse wave information, the event determination unit determines that the syncope event has occurred in the subject.

5. The biological information detecting device according to claim 4,
wherein, when a pulse rate specified based on the pulse wave information is equal to or greater than a second threshold value and a pulse wave amplitude at a first timing specified based on the pulse wave information is greater than a pulse wave amplitude at a second timing after the first timing, the event determination unit determines that the subject is in the tachycardia state.

6. The biological information detecting device according to claim 1,
wherein, when it is determined that the subject is in a pulse missing state based on the pulse wave information, the event determination unit determines that the syncope event has occurred in the subject.

7. The biological information detecting device according to claim 6,
wherein, when a pulse rate specified based on the pulse wave information is equal to or greater than a third threshold value and less than a fourth threshold value and no pulse is detected over a given period of time in a pulse waveform specified based on the pulse wave information, the event determination unit determines that the subject is in the pulse missing state.

8. The biological information detecting device according to claim 1,
wherein, when it is determined that the subject is in an atrial fibrillation state based on the pulse wave information, the event determination unit determines that the syncope event has occurred in the subject.

9. The biological information detecting device according to claim 1,
wherein, when it is determined that blood oxygen saturation of the subject is equal to or less than a given set value, the event determination unit determines that the syncope event has occurred in the subject.

10. The biological information detecting device according to claim 1,
wherein, when it is determined that the subject has fallen down due to syncope based on body motion sensor information obtained from a body motion sensor, the event determination unit determines that the syncope event has occurred in the subject.

11. The biological information detecting device according to claim 1,
wherein the pulse wave information at the time of occurrence of the syncope event includes information of at least one of a pulse rate at the time of occurrence of the syncope event and a pulse waveform at the time of occurrence of the syncope event.

12. The biological information detecting device according to claim 1,
wherein the control unit outputs the stored pulse wave information at the time of occurrence of the syncope event as notification information that is notified by a notification unit.

13. The biological information detecting device according to claim 1,
wherein the control unit calculates autonomic nervous activity information of the subject based on the pulse wave information, associates the autonomic nervous activity information with the pulse wave information at the time of occurrence of the syncope event, and outputs the autonomic nervous activity information as notification information that is notified by the notification unit.

14. The biological information detecting device according to claim 1,
wherein the event determination unit determines whether or not the syncope event has occurred based on reference pulse wave information for determination.

15. The biological information detecting device according to claim 14,
wherein the reference pulse wave information is the pulse wave information corresponding to a timing of receiving an operation of a user.

16. The biological information detecting device according to claim 14,
wherein the event determination unit determines whether or not the syncope event has occurred by comparing the reference pulse wave information with the pulse wave information measured by the pulse wave information measuring unit.

17. A biological information detecting device, comprising:
a pulse wave information measuring unit that measures pulse wave information of a subject; and
a control unit that calculates syncope index information, which changes according to a situation of a sign of syncope or development of syncope in the subject, based on the measured pulse wave information,
wherein the control unit calculates the syncope index information based on pulse wave amplitude information of a plurality of pulsations within a given period of time.

18. The biological information detecting device according to claim 17,
wherein the control unit calculates the syncope index information based on a total value of the pulse wave amplitude information of the plurality of pulsations within the given period of time.

19. The biological information detecting device according to claim 18,
wherein the control unit calculates the syncope index information by performing multiplication processing between the total value and a coefficient for calculation of the syncope index information.

20. The biological information detecting device according to claim 19,
wherein the control unit calculates the syncope index information by performing the multiplication processing between the total value and the coefficient that changes according to a pulse rate of the subject.

21. The biological information detecting device according to claim 20,
wherein the control unit calculates the syncope index information by performing the multiplication processing between the total value and the coefficient that increases in a first pulse rate range and decreases in a second pulse rate range having the higher pulse rate than in the first pulse rate range.

22. The biological information detecting device according to claim 21,
wherein the control unit calculates the syncope index information by performing the multiplication processing between the total value and the coefficient that is a fixed value in a third pulse rate range between the first and second pulse rate ranges.

23. The biological information detecting device according to claim 17,
wherein the control unit outputs the calculated syncope index information as notification information that is notified by a notification unit.

24. The biological information detecting device according to claim 23,
wherein the control unit outputs information obtained by associating the pulse wave information, from which the syncope index information has been calculated, with the syncope index information as notification information that is notified by the notification unit.

25. The biological information detecting device according to claim 23,
wherein the control unit outputs the syncope index information at the time of occurrence of an event and the syncope index information in a normal state as the notification information that is notified by the notification unit.
